(19) United States
(12) Patent Application Publication
(10) Pub. No.: US 2010/0111992 A1
(43) Pub. Date: May 6, 2010

(54) CYTOMEGALOVIRUS PEPTIDES AND METHODS OF USE THEREOF

(75) Inventors: Salvatore Albani, Encinitas, CA (US); Adriana H. TTremoulet, San Diego, CA (US)

Correspondence Address: DLA PIPER LLP (US), 4365 EXECUTIVE DRIVE, SUITE 1100, SAN DIEGO, CA, 92121-2133, US

(73) Assignee: THE REGENTS OF THE UNIVERSITY OF CALIFORNIA, Oakland, CA (US)

(21) Appl. No.: 12/297,907

(22) PCT Filed: May 1, 2007
(86) PCT No.: PCT/US2007/010658

§ 371 Date: Jan. 14, 2010

Related U.S. Application Data
(60) Provisional application No. 60/796,670, filed on May 1, 2006.

(51) Int. Cl. A61K 39/12; C07K 2/00; C12N 15/11; C12N 15/33; C12N 15/19; C12N 15/85; C12N 5/10; A61K 35/12; C12Q 1/70

(52) U.S. Cl. 424/186.1; 530/350; 536/23.1; 530/403; 536/23.5; 435/320.1; 435/325; 424/93.7; 435/5

(57) ABSTRACT

A method of modulating an immune response in a subject is disclosed. The invention is based on the discovery that an effective therapeutic strategy for ameliorating the symptoms of cytomegalovirus infection can be achieved by administering an effective amount of a CMV-derived peptide.

### BACKGROUND OF THE INVENTION

1. Field of the Invention

The present invention relates generally to methods and compositions for modulating an immune response in a subject, and more specifically to compositions containing a cytomegalovirus peptide and methods of using such compositions to stimulate an immune response or to generate tolerance to an immunogen.

2. Background Information

Vertebrates possess the ability to mount an immune response as a defense against pathogens from the environment as well as against aberrant cells, such as tumor cells, which develop internally. This can take the form of innate immunity, which is mediated by NK cells, neutrophils, and cells of the monocyte/macrophage lineage, or the form of acquired or active immunity against specific antigens, which is mediated by lymphocytes. Active immune responses can be further subdivided into two arms, the humoral response, which entails the production of specific antibodies that serve to neutralize antigens exposed to the systemic circulation and aid in their uptake by professional phagocytic cells, and the cellular arm, which is responsible for the recognition of infected or aberrant cells within the body. Often these immunogenic responses result in diseases and disorders that cause harm to the organism itself. Such disorders are associated with the recognition of self proteins and cells as foreign, and thus trigger an attack upon such cells or self proteins. Common autoimmune disorders include, for example, psoriasis, arthritis, lupus, diabetes, and other medical conditions known in the art.

Treatment strategies for many diseases are directed at alleviating the symptoms of the disease rather than resolving the cause of the problematic symptoms. Congenital CMV, or cytomegalovirus, is the most common congenital (present at birth) infection in the United States. Although primary infection with this agent generally does not produce symptoms in healthy adults, several high-risk groups, including immunocompromised organ transplant recipients and individuals infected with HIV, are at risk of developing life- and sight-threatening CMV disease. In addition, CMV has emerged in recent years as the most important cause of congenital infection in the developed world, commonly leading to mental retardation and developmental disability.

Most CMV infections are “silent,” meaning they cause no signs or symptoms in an infected person. However, CMV can cause disease in unborn babies and in people with a weakened immune system. Transmission of CMV occurs from person to person, through close contact with body fluids (urine, saliva (spit), breast milk, blood, tears, semen, and vaginal fluids), but the chance of getting CMV infection from casual contact is very small. In the United States, about 1%-4% of uninfected mothers have primary (or first) CMV infection during a pregnancy. 33% of women who become infected with CMV for the first time during pregnancy pass the virus to their unborn babies.

In 1904, Ribbert first identified histopathological evidence of CMV probably in tissues from a congenitally infected infant. Ribbert mistakenly assumed that the large inclusion-bearing cells he observed at autopsy were from protozoa (incorrectly named Entamoeba mortinatalium). In 1920, Goodpasture correctly postulated the viral etiology of these inclusions. Goodpasture used the term cytomegalia to refer to the enlarged, swollen nature of the infected cells. Human CMV (HCMV) was first isolated in tissue culture in 1956, and the propensity of this organism to infect the salivary gland led to its initial designation as a salivary gland virus.

In 1960, Weller designated the virus cytomegalovirus, and during the 1970s and 1980s, knowledge of the role of CMV as an important pathogen with diverse clinical manifestations increased steadily.

Ganciclovir has been shown to have antiviral activity in vitro and in vivo against various Herpesviridae. Its principal use has been against CMV, but it has been active against HSV-1 & -2, HHV type 6, VZV and EBV. Ganciclovir interferes with DNA synthesis and inhibits viral replication of susceptible viruses. The antiviral activity depends on intracellular conversion of the drug to ganciclovir triphosphate.

Although enormous progress has recently been made in defining and characterizing the molecular biology, immunology, and antiviral therapeutic targets for CMV, considerable work remains in devising strategies for prevention of CMV infection. There exists for a need for a therapy for patients suffering from cytomegalovirus infection who have failed ganciclovir therapy and other current therapies. The need extends to patients who would be at great risk for cytomegalovirus reactivation, such as transplant patients who are CMV+ or who have a CMV+ donor.

### SUMMARY OF THE INVENTION

The invention is based on the discovery that an effective therapeutic strategy for ameliorating the symptoms of cytomegalovirus infection can be achieved by administering an effective amount of a CMV-derived peptide. As such, the present invention relates to CMV-derived peptides, and methods of using such peptides for modulating an immune response in a subject.

Accordingly, the present invention provides methods of modulating an immune response in a subject having or at risk of having cytomegalovirus infection by administering a CMV-derived peptide to the subject, thereby modulating an immune response in the subject. In one embodiment, the CMV-derived peptides of the invention are administered directly to the subject. In another embodiment, a sample of cells from the subject are contacted ex vivo with an isolated peptide selected from any one of SEQ ID NOS: 1-16 and any combination thereof, and are subsequently administered to the subject, thereby stimulating an immune response to the cytomegalovirus infection in the subject.

The CMV-derived peptide can be any immunogenic portion of human or murine CMV pp65 and ppM83, respectively, including a glycosylated form of such a peptide, and generally is a peptide that can bind an MHC class II receptor or a T cell receptor, and that provides an epitope that induces a proinflammatory immune response in human T cell effectors. For example, the peptide can be any peptide having the amino acid sequence RVVMPRTVQLRTGQS (SEQ ID NO: 1); TVQLRTGQSVVLTST (SEQ ID NO: 2); GFRVVMPRTVQLRTG (SEQ ID NO: 3); RNLVRAATRDAMGAA (SEQ ID NO: 4); NSVKVDASAVRQASV (SEQ ID NO: 5); ATTTRTGMKTVRMTV (SEQ ID NO: 6); RLLQTGIHV (SEQ ID NO: 7); ALPLKMNLNI (SEQ ID NO: 8); YTSAFVFPT (SEQ ID NO: 9); VLCPKNMII (SEQ ID NO: 10); MSIYVYALPLKMLNI (SEQ ID NO: 11); MISVLGPISGHVLKA (SEQ ID NO: 12); HVRVSQPSLILVSQY (SEQ ID NO: 13); DVYYTSAFVFPTKDV (SEQ ID NO: 14); SGKLFMHVTLGSDVE (SEQ ID NO: 15); or AGILARNLVPMVATV (SEQ ID NO: 16), or any combination thereof.

The invention also provides a method for identifying an agent that enhances the enhances stimulation of an immune response in a subject having or at risk of having cytomegalovirus infection by contacting a sample comprising cells that express a detectable marker with a test agent and an isolated peptide selected from any one of SEQ ID NOS: 1-16. Any increase in the expression of the detectable marker in the presence of the agent as compared with expression of the detectable marker in the absence of the agent is indicative of an agent that enhances stimulation of an immune response in a subject having or at risk of having cytomegalovirus infection. Markers for use in the methods of the invention include, but are not limited to CD69, TNFα, IFNγ, and IL-2.

### BRIEF DESCRIPTION OF THE DRAWINGS

FIG. 1 is a graphical diagram showing a proliferation assay of CD4+ induced cells.

FIGS. 2A-2C are graphical diagrams showing data that peptides 1DR and 4DR stimulate expression of IL-2 in enriched CD4+ cells, as compared to no peptide.

FIGS. 3A and 3B are graphical diagrams showing data from a proliferation assay of CD4 induced cells in donors of different HLA alleles. FIG. 3A shows Donor SA005 HLA DRB1 1/1, and FIG. 3B shows Donor SA006 HLA DRB1 1/11.

FIGS. 4A and 4B are graphical diagrams showing that peptides 1DR to 4DR stimulate TNFα synthesis as compared to control. FIG. 4A shows Donor SA005 HLA DRB1 1/1, while FIG. 4B shows Donor SA006 HLA DRB1 1/11.

FIGS. 5A and 5B are graphical diagrams showing the immunogenicity of peptides 1DR to 4DR in two healthy CMV+ donors. FIG. 5A shows Donor SA005 HLA DRB1 1/1, while FIG. 5B shows Donor SA006 HLA DRB1 1/11.

FIGS. 6A and 6B are graphical diagrams showing the immunogenicity of peptides 1DR to 4DR in four healthy CMV+ donors.

FIGS. 7A and 7B are graphical diagrams showing that the immunogenicity (reflected by increased TNFα) of peptides 1DR, 3DR and 4DR in a CMV-infected subject (CMV001) is greater than in a healthy CMV+ subject (BB008). FIG. 7A shows Donor BB008, while FIG. 7B shows Donor CMV001.

FIGS. 8A and 8B are graphical diagrams showing that the immunogenicity (reflected by increased IFNg) of peptides 1DR, 3DR and 4DR in a CMV-infected subject (CMV001) is greater than in a healthy CMV+ subject (BB008). FIG. 8A shows Donor BB008, while FIG. 8B shows Donor CMV001.

FIGS. 9A and 9B are graphical diagrams showing that there is more surface expression of PD-1 (programmed cell death-1) with peptides 1DR, 3DR and 4DR in a CMV-infected subject (CMV001) is greater than in a healthy CMV+ subject (BB008). This correlates with latency versus infection in the two subjects. FIG. 9A shows Donor BB008, while FIG. 9B shows Donor CMV001.

FIGS. 10A and 10B are graphical diagrams showing that there is more surface expression of PD-L1 (a PD-1 ligand) with peptides 1DR, 4DR, 5DR and 6DR in a CMV-infected subject (CMV001) is greater than in a healthy CMV+ subject (BB008). FIG. 10A shows Donor BB008, while FIG. 10B shows Donor CMV001.

FIGS. 11A and 11B are graphical diagrams showing that there is more surface expression of PD-L2 (a PD-1 ligand) with peptides 1DR, 4DR, 5DR and 6DR in a CMV-infected subject (CMV001) is greater than in a healthy CMV+ subject (BB008). FIG. 11A shows Donor BB008, while FIG. 11B shows Donor CMV001.

FIGS. 12A and 12B are graphical diagrams showing that the immunogenicity of peptides 1DR, 3DR, and 4DR (reflected by increased TNFα and IFNg) in an experiment with six donors, excluding CMV-infected subject (CMV001).

FIGS. 13A and 13B are graphical diagrams showing that the immunogenicity of peptides 1 DR and 4DR (reflected by increased TNFα and IFNg) in an experiment with six donors, including CMV-infected subject (CMV001).

FIGS. 14A and 14B show data from a proliferation assay of cells from various donors using methods known in the art. FIG. 14A shows cells from donor BB008, while FIG. 14B shows cells from donor CMV001.

FIGS. 15A and 15B show data from a proliferation assay of cells from various donors using methods known in the art. FIG. 15A shows cells from donor BB008 with standard peptides 1DR-6DR and with Ova peptide, while FIG. 15B shows cells from donor CMV001 with standard peptides 1DR-6DR and with Ova peptide.

FIGS. 16A and 16B show data from a proliferation assay of cells from various donors using methods known in the art. FIG. 16A shows cells from donor BB011 with peptides 1DR-6DR, while FIG. 16B shows cells from donor BB013 with peptides 1DR-6DR.

FIGS. 17A and 17B show data from a proliferation assay of cells from various donors using methods known in the art. FIG. 17A shows cells from donor BB011 with standard peptides 1DR-6DR and with Ova peptide, while FIG. 17B shows cells from donor BB013 with standard peptides 1DR-6DR and with Ova peptide.

FIG. 18 shows data from a proliferation assay of cells from various donors using methods known in the art. Data from the six donors SA001, SA005, SA006, BB006, BB008, CMV001, BB011, and BB013 are shown. No peptide versus 3DR (P<0.001); no peptide versus 4DR (P<0.05); 1DR versus 3DR (P<0.05); 2DR versus 3DR (P<0.01); and 3DR versus 6DR (P<0.05).

### DETAILED DESCRIPTION OF THE INVENTION

The invention provides a method of modulating an immune response in a subject. As disclosed herein, an effective therapeutic strategy for ameliorating the symptoms of cytomegalovirus (CMV) infection can be achieved by modulating the underlying immune response.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

CMV is a member of the family of 8 human herpesviruses, designated as human herpesvirus 5 (HHV-5). Taxonomically, CMV is referred to as a Betaherpesvirinae, based on its propensity to infect mononuclear cells and lymphocytes and on its molecular phylogenetic relationship to other herpesviruses. CMV is the largest member of the herpesvirus family, with a double-stranded DNA genome of more than 240 kbp, capable of encoding more than 200 potential protein products. The function of most of these proteins remains unclear. As with the other herpesviruses, the structure of the viral particle is that of an icosahedral capsid, surrounded by a lipid bilayer outer envelope.

CMV replicates very slowly in cell culture, mirroring its very slow pattern of growth in vivo (in contrast to herpes simplex virus (HSV) infection, which progresses very rapidly). The replication cycle of CMV is divided temporally into the following 3 regulated classes: immediate early, early, and late.

Immediate early gene transcription occurs in the first 4 hours following viral infection, when key regulatory proteins, which allow the virus to take control of cellular machinery, are made. The major immediate early promoter of this region of the CMV genome is one of the most powerful eukaryotic promoters described in nature, and this has been exploited in modern biotechnology as a useful promoter for driving gene expression in gene therapy and vaccination studies.

Following synthesis of immediate early genes, the early gene products are transcribed. Early gene products include DNA replication proteins and some structural proteins.

Finally, the late gene products are made approximately 24 hours after infection, and these proteins are chiefly structural proteins that are involved in virion assembly and egress. Synthesis of late genes is highly dependent on viral DNA replication and can be blocked by inhibitors of viral DNA polymerase, such as ganciclovir (GCV). The lipid bilayer outer envelope contains the virally encoded glycoproteins, which are the major targets of host neutralizing antibody responses. These glycoproteins are candidates for human vaccine design. The proteinaceous layer between the envelope and the inner capsid, the viral tegument, contains proteins that are major targets of host cell-mediated immune responses.

In clinical specimens, one of the classic hallmarks of CMV infection is the cytomegalic inclusion cell. These massively enlarged cells (the property of cytomegaly from which CMV acquires its name) contain intranuclear inclusions, which histopathologically have the appearance of owl's eyes. The presence of these cells indicates productive infection, although they may be absent even in actively infected tissues. In most cell lines, CMV is difficult to culture in the laboratory, but in vivo infection seems to involve chiefly epithelial cells, and, with severe disseminated CMV disease, involvement can be observed in most organ systems.

Although the CNS is the major target organ for tissue damage in the developing fetus, culturing CMV from the cerebrospinal fluid of symptomatic congenitally infected infants is surprisingly difficult. Because CMV can infect endothelial cells, some authors have postulated that a viral angitis may be responsible for perfusion failure of developing brain with resultant maldevelopment. Others have postulated a direct teratogenic effect of CMV on the developing fetus. Observation of CMV-induced alternations in the cell cycle and CMV-induced damage to chromosomes supports this speculation.

Immunity to CMV is complex and involves humoral and cell-mediated responses. Several CMV gene products are of particular importance in CMV immunity. The outer envelope of the virus, which is derived from the host cell nuclear membrane, contains multiple virally encoded glycoproteins. Glycoprotein B (gB) and glycoprotein H (gH) appear to be the major determinants of protective humoral immunity. Antibody to these proteins is capable of neutralizing virus, and gB and gH are targets of investigational CMV subunit vaccines; however, although humoral responses are important in control of severe disease, they are clearly inadequate in preventing transplacental infection, which can occur even in women who are CMV-seropositive.

The generation of cytotoxic T-cell (CTL) responses against CMV may be a more important host immune response in control of infection. In general, these CTLs involve major histocompatibility complex (MHC) class I restricted CD8+ responses. Although many viral gene products are important in generating these responses, most CMV-specific CTLs target an abundant phosphoprotein in the viral tegument, pp65, the product of the CMV UL83 gene. In passive transfer experiments involving high-risk bone marrow transplant recipients, the value of these responses was dramatically demonstrated using adoptive transfer of CMV-specific CD8+ T cells that target the CMV UL83 gene, which was able to control CMV disease.

Recent investigations into the molecular biology of CMV have revealed the presence of many viral gene products, which appear to modulate host inflammatory and immune responses. As used herein, the term “modulate” means “increase” or “reduce or inhibit.” The terms “increase” and “reduce or inhibit” are used in reference to a baseline level of a specified activity or response (e.g., host inflammatory and immune responses), which can be the level of the specified activity or response in the absence of an agent that has the modulating activity, or the level of the specified activity with respect to a corresponding normal cell. For example, the methods of the invention are useful for modulating (e.g., increasing or stimulating) an immune response in a subject having or at risk of having cytomegalovirus infection. As such, in one embodiment, the methods for modulating an immune response include administering to the subject an isolated peptide selected from any one of SEQ ID NOS: 1-16, or any combination thereof, wherein the administration modulates an immune response to the cytomegalovirus infection in the subject.

As used herein, the term “immunoeffector cells” refers to cells that are directly involved in generating or effecting an immune response. Such cells are well known in the art and include B lymphocytes (B cells); antigen presenting cells such as dendritic cells, mononuclear phagocytic cells, macrophages, including Langerhans cells and, in humans, venular endothelial cells (and B cells); and, particularly T cells, for example, T helper cells, T suppressor cells, and cytotoxic T cells.

As used herein, the term “immunizing conditions” means that a peptide of the invention is contacted with a cell or administered to a subject such that it can effect its immunogenic activity. As such, the peptide, which is a T cell immunogen, generally will be administered in an immunogenic amount, typically as a priming dose followed some time later by one or more booster doses, intradermally, subcutaneously, or intramuscularly, and, if desired, formulated in a composition that includes an immunoadjuvant such as Freund's complete or incomplete adjuvant.

As used herein, the term “tolerizing conditions” means that a peptide of the invention is contacted with a cell or administered to a subject such that it induces tolerization to the otherwise immunogenic activity. As a result, a subject, for example, is tolerized to the peptide such that it is recognized as “self” by the subject and cannot effect an immune response. A peptide can be administered under tolerizing conditions by administering a tolerizing amount of the peptide, generally a small amount over a period of time, intradermally, subcutaneously, intramuscularly, or, preferably, mucosally, for example, via nasal spray or by eating.

As used herein “corresponding normal cells” means cells that are from the same organ and of the same type as the disorder or disease examined. In one aspect, the corresponding normal cells comprise a sample of cells obtained from a healthy individual. Such corresponding normal cells can, but need not be, from an individual that is age-matched and/or of the same sex as the individual providing the sample containing CMV being examined.

In another aspect, the present invention provides a method of ameliorating or treating a subject having or at risk of having CMV infection. In one embodiment, one or more peptides of the invention are administered to the subject, thereby ameliorating the signs and/or symptoms associated with CMV infection. In a related embodiment, cells are contacted ex vivo and subsequently administered to the subject. Thus, CMV-specific cells may be generated ex vivo using one or more of the peptides or polynucleotides encoding the peptides of the invention, and thereafter infused into the subject, thereby ameliorating the signs and/or symptoms associated with CMV infection. Methods for transfecting cells and tissues removed from an organism in an ex vivo setting are known to those of skill in the art. Thus, it is contemplated that cells or tissues may be removed and transfected ex vivo using the nucleic acids of the present invention.

As used herein, the term “ameliorating” or “treating” means that the clinical signs and/or the symptoms associated with CMV infection are lessened as a result of the actions performed. Examples of clinical signs and/or symptoms associated with CMV infection include, but are not limited to, fever, hepatosplenomegaly (enlarged liver and spleen), mental or motor retardation, malaise, and muscle and joint pain but without sore throat, and sometimes death. The signs or symptoms to be monitored will be characteristic of CMV infection and will be well known to the skilled clinician, as will the methods for monitoring the signs and conditions.

Thus, the symptoms of CMV infection vary depending upon the age and health of the person who is infected, and how the infection occurred. Infants who are infected before birth usually show no symptoms of a CMV infection after they are born, although some of these infants can develop hearing, vision, neurologic, and developmental problems over time. In a few cases, there are symptoms at birth, which can include premature delivery, being small for gestational age, jaundice, enlarged liver and spleen, microcephaly (small head), seizures, rash, and feeding difficulties. These infants are also at high risk for developing hearing, vision, neurologic, and developmental problems.

Several CMV genes interfere with normal antigen processing and generation of cell-mediated immune responses. To date, three viral gene products have been identified that inhibit MHC class I antigen presentation. One is the US11 gene product, which exports the class I heavy chain from the endoplasmic reticulum (ER) to the cytosol (rendering it nonfunctional). Another is the US3 gene product, which retains MHC molecules in the ER, preventing them from traveling to the plasma membrane. Finally, the US6 protein inhibits peptide translocation by transporters associated with antigen processing (TAP).

Other viral gene products, the UL33, US27, and US28 genes, are functional homologs of cellular G-protein coupled receptors which may, via molecular mimicry, subvert normal inflammatory responses and, in the process, promote tissue dissemination of the virus and interfere with host immune response. The CMV genome also encodes a homolog of the cellular major histocompatibility class I gene, which appears to contribute to the ability of CMV to evade host defense. The UL144 open reading frame found in clinical isolates of CMV encodes a structural homolog of the tumor necrosis factor receptor superfamily, which may contribute to the ability of HCMV to escape immune clearance.

Accordingly, the invention relates to the collection of human and murine CMV pp65- and ppM83-derived peptides, respectively, selected for their immunogenicity based on available matrix algorithms. These peptides were chosen using matricies that have been validated by comparison to MHC binding assays. Furthermore, the matricies are based on HLA superfamilies and, therefore predict peptides that bind well to a number of HLA polymorphisms.

The term “protein” or “peptide” as used herein, refers to at least two covalently attached amino acids, which includes proteins, polypeptides, oligopeptides and peptides. A protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration.

The term “nucleic acid” or “oligonucleotide” or grammatical equivalents as used herein, refers to at least two nucleotides covalently linked together. A nucleic acid will generally contain phosphodiester bonds, although in some cases, as outlined below, nucleic acid analogs are included that may have alternate backbones, comprising, for example, phosphoramide (Beaucage, et al., Tetrahedron, 49(10):1925 (1993) and references therein; and Pauwels, et al., Chemica Scripta, 26:141 (1986)), phosphorothioate (Mag, et al., Nucleic Acids Res., 19:1437 (1991); and U.S. Pat. No. 5,644,048), phosphorodithioate (Briu, et al., J. Am. Chem. Soc., 111:2321 (1989)), O-methylphophoroamidite linkages (see Eckstein, Oligonucleotides and Analogues: A Practical Approach, Oxford University Press), and peptide nucleic acid backbones and linkages. Other analog nucleic acids include those with positive backbones, non-ionic backbones and non-ribose backbones, including those described in U.S. Pat. Nos. 5,235,033 and 5,034,506. Nucleic acids containing one or more carbocyclic sugars are also included within the definition of nucleic acids (see Jenkins, et al., Chem. Soc. Rev., (1995) pp. 169-176). All of these references are hereby expressly incorporated by reference. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc.

The peptides of the invention that have been isolated are identified in Table 1.

TABLE 1

| | | |
|---|---|---|
| Murine (IAd) | RVVMPRTVQLRTGQS | SEQ ID NO: 1 |
| | TVQLRTGQSVVLTST | SEQ ID NO: 2 |
| | GFRVVMPRTVQLRTG | SEQ ID NO: 3 |
| | RNLVRAATRDAMGAA | SEQ ID NO: 4 |
| | NSVKVDASAVRQASV | SEQ ID NO: 5 |
| | ATTTRTGMKTVRMTV | SEQ ID NO: 6 |
| Human (A2) | RLLQTGIHV | SEQ ID NO: 7 |
| | ALPLKMNLNI | SEQ ID NO: 8 |
| | YTSAFVFPT | SEQ ID NO: 9 |
| | VLCPKNMII | SEQ ID NO: 10 |
| Human (DRB1) | MSIYVYALPLKMLNI | SEQ ID NO: 11 |
| | MISVLGPISGHVLKA | SEQ ID NO: 12 |
| | HVRVSQPSLILVSQY | SEQ ID NO: 13 |
| | DVYYTSAFVFPTKDV | SEQ ID NO: 14 |
| | SGKLFMHVTLGSDVE | SEQ ID NO: 15 |
| | AGILARNLVPMVATV | SEQ ID NO: 16 |

As such, these experiments focus on confirming that the in vitro recognition of the novel class I and class II CMV epitopes predicted to be strong MHC binders by validated computer algorithms using T cells from CMV-positive healthy donors. Phenotypical analysis of CD4+ T cell effectors included a marker of activation (CD69) as well as T cell inflammatory cytokines (TNFα, IFNγ, and IL-2). Functional analysis of CD4+ and CD8+ T cell effectors was measured by proliferative epitope-specific responses via ³H incorporation and IFNγ expression measured by ELISA, respectively.

The term “cytokine” is used broadly herein to refer to soluble glycoproteins that are released by cells of the immune system and act non-enzymatically through specific receptors to regulate immune responses. As such, the term “cytokine” as used herein includes chemokines, interleukins, lymphokines, monokines, interferons, colony stimulating factors, platelet activating factors, tumor necrosis factor-alpha, and receptor associated proteins, as well as functional fragments thereof.

To induce an immune response to CMV infection in a subject, the antigenicity of the protein or peptide may be enhanced by coupling it to a carrier protein by conjugation using techniques which are well-known in the art. Such commonly used materials which are chemically coupled to the molecule to enhance their antigenicity include keyhole limpet hemocya-nin (KLH), thyroglobulin, bovine serum albumin (BSA), and tetanus toxoid. The coupled molecule is then used to immunize the subject. Thus, in one aspect, the invention provides a method for identifying an agent that enhances the enhances stimulation of an immune response in a subject having or at risk of having cytomegalovirus infection by contacting a sample comprising cells that express a detectable marker with a test agent and an isolated peptide selected from any one of SEQ ID NOS: 1-16. Any increase in the expression of the detectable marker in the presence of the agent as compared with expression of the detectable marker in the absence of the agent is indicative of an agent that enhances stimulation of an immune response in a subject having or at risk of having cytomegalovirus infection. Markers for use in the methods of the invention include, but are not limited to CD69, TNFα, IFNγ, and IL-2.

In another aspect, the invention provides methods for identifying CMV-derived peptides for use in the methods of the invention. As such, sequencing algorithms can be used to measure sequence identity between known and unknown sequences. Such methods and algorithms are useful in identifying corresponding sequences present in other organisms as well as in the design of peptides of the invention. Sequence identity is often measured using sequence analysis software (e.g., Sequence Analysis Software Package of the Genetics Computer Group, University of Wisconsin Biotechnology Center, 1710 University Avenue, Madison, Wis. 53705). Such software matches similar sequences by assigning degrees of identity to various deletions, substitutions, and other modifications. The terms “homology” and “identity” in the context of two or more nucleic acids, polypeptides, or peptide sequences refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection.

One example of a useful algorithm is BLAST and BLAST 2.0 algorithms, which are described by Altschul et al. (Nucleic Acids Res. 25:3389-3402, 1977; J. Mol. Biol. 215:403-410, 1990, each of which is incorporated herein by reference). Software for performing BLAST analyses is publicly available through the National Center for Biotechnology Information (available on the world wide web at the URL ncbi.nlm.nih.gov). This algorithm involves first identifying high scoring sequence pairs by identifying short words of length W in the query sequence, which either match or satisfy some positive-valued threshold score T when aligned with a word of the same length in a database sequence. T is referred to as the neighborhood word score threshold (Altschul et al., supra, 1977, 1990). These initial neighborhood word hits act as seeds for initiating searches to find longer high scoring sequence pairs containing them. The word hits are extended in both directions along each sequence for as far as the cumulative alignment score can be increased. Cumulative scores are calculated using, for nucleotide sequences, the parameters M+ (reward score for a pair of matching residues; always >0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=4 and a comparison of both strands. For amino acid sequences, the BLASTP program uses as defaults a wordlength of 3, and expectations (E) of 10, and the BLOSUM62 scoring matrix (see Henikoff and Henikoff, Proc. Natl. Acad. Sci., USA 89:10915, 1989) alignments (B) of 50, expectation (E) of 10, M=5, N=−4, and a comparison of both strands.

The BLAST algorithm also performs a statistical analysis of the similarity between two sequences (see, for example, Karlin and Altschul, Proc. Natl. Acad. Sci., USA 90:5873, 1993, which is incorporated herein by reference). One measure of similarity provided by BLAST algorithm is the smallest sum probability (P(N)), which provides an indication of the probability by which a match between two nucleotide or amino acid sequences would occur by chance. For example, a nucleic acid is considered similar to a references sequence if the smallest sum probability in a comparison of the test nucleic acid to the reference nucleic acid is less than about 0.2, more preferably less than about 0.01, and most preferably less than about 0.001.

In one embodiment, protein and nucleic acid sequence homologies are evaluated using the Basic Local Alignment Search Tool (“BLAST”). In particular, five specific BLAST programs are used to perform the following task:

(1) BLASTP and BLAST3 compare an amino acid query sequence against a protein sequence database;

(2) BLASTN compares a nucleotide query sequence against a nucleotide sequence database;

(3) BLASTX compares the six-frame conceptual translation products of a query nucleotide sequence (both strands) against a protein sequence database;

(4) TBLASTN compares a query protein sequence against a nucleotide sequence database translated in all six reading frames (both strands); and

(5) TBLASTX compares the six-frame translations of a nucleotide query sequence against the six-frame translations of a nucleotide sequence database.

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. Preferably, the scoring matrix used is the BLOSUM62 matrix (Gonnet et al., Science 256:1443-1445, 1992; Henikoff and Henikoff, Proteins 17:49-61, 1993, each of which is incorporated herein by reference). Less preferably, the PAM or PAM250 matrices may also be used (Schwartz and Dayhoff, eds., “Matrices for Detecting Distance Relationships: Atlas of Protein Sequence and Structure” (Washington, National Biomedical Research Foundation 1978)). BLAST programs are accessible through the U.S. National Library of Medicine, for example, on the world wide web at the URL ncbi.nlm.nih.gov. The parameters used with the above algorithms may be adapted depending on the sequence length and degree of homology studied. In some embodiments, the parameters may be the default parameters used by the algorithms in the absence of instructions from the user.

A peptide of the invention can be prepared using methods of chemical peptide synthesis, can be expressed from an encoding polynucleotide, or isolated by methods known in the art. Techniques for purifying, synthesizing or producing peptides in recombinant form are convenient and well known in the art, and are suitable for producing immunogenic peptides of sufficient purity for use in a method of the invention. In this respect, the term “isolated” or “substantially pure” denotes a polypeptide or polynucleotide that is substantially free of other compounds with which it may normally be associated in vivo. In the context of a method of the invention, the term substantially pure refers to substantially homogenous peptides or polynucleotides, where homogeneity is determined by reference to purity standards known in the art such as purity sufficient to allow the N-terminal amino acid sequence of the protein to be obtained. Preferably, the peptide or polynucleotide is sufficiently isolated such that it can be used for administration to a subject. As such, an isolated peptide or polypeptide generally constitutes at least about 50% of a sample containing the peptide, usually at least about 75%, particularly at least about 90%, and preferably about 95% to 99% or more. It should be recognized that such a measure of purity refers to the peptide alone, or as a starting material, for example, for formulation into a composition, in which case the isolated peptide of the invention can comprise a component of the composition, which can further contain additional components as disclosed herein, including additional isolated peptides of the invention.

As used herein, the term “functional fragment” refers to a peptide or polypeptide portion of a protein that possesses the biological function or activity characteristic of the native protein. For example, a functional fragment of IFNγ or TNFα has, for example, substantially the same pro-inflammatory activity as naturally occurring or recombinantly produced IFNγ or TNFα, respectively.

The term “subject” as used herein refers to any individual or patient to which the invention methods are performed. For example, a subject may be any one having or at risk of having cytomegalovirus infection. Generally the subject is human, although as will be appreciated by those in the art, the subject may be an animal.

The terms “sample” and “biological sample” as used herein, refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., serum, plasma, saliva, urine, and ejaculate.

The present invention also provides a polynucleotide encoding an immunogenic peptide of the invention. The polynucleotide can be single stranded or double stranded, and can be a ribonucleic acid molecule (RNA), a deoxyribonucleic acid molecule (DNA), or a hybrid thereof. In addition, the invention provides a recombinant nucleic acid molecule, which includes a polynucleotide of the invention operatively linked to at least one heterologous nucleotide sequence. The heterologous nucleotide sequence can be any nucleotide sequence that is not normally found in contiguous linkage with the polynucleotide of the invention in nature. For example, the heterologous nucleotide sequence can be an expression control sequence such as a transcription regulatory element or a translation regulatory element, or a combination thereof; or can encode a polypeptide such as a cytokine or other immunomodulatory agent, a peptide tag, a cellular localization domain, or the like. Where the recombinant nucleic acid molecule encodes a peptide of the invention and a second (or more) functional polypeptide such as one or more additional peptides of the invention or one or more cytokines or the like, the recombinant nucleic acid molecule can further encode a protease recognition site between each of the encoded peptides such that, upon expression, each of the encoded peptides is released in a form that is free from the other encoded peptides or polypeptides.

The present invention also provides a vector containing a polynucleotide of the invention, and further provides a cell that contains a polynucleotide or vector of the invention. The vector can be a cloning vector, which can be useful for producing a large amount of polynucleotide or recombinant nucleic acid molecule of the invention contained therein, or can be an expression vector, which can be useful if the polynucleotide is to be administered to a cell or subject for the purpose of expressing the encoded peptide. Such vectors are well known in the art and include, for example, plasmid vectors and viral vectors, including vectors derived from a retrovirus, adenovirus, adeno-associated virus, vaccinia virus or the like.

A commonly used plasmid vector which operatively encodes foreign structural gene inserts is the pBR322 plasmid. pBR322 includes a gene for conferring ampicillin resistance as a marker; however, for use in humans, such ampicillin resistance should be avoided. Modified vectors which are useful in gene immunization protocols but do not confer ampicillin resistance are described, for example, in U.S. Ser. No. 08/593,554, filed Jan. 30, 1996, which is incorporated herein by reference.

Various viral vectors that can be utilized in the invention include adenovirus, herpes virus, vaccinia, or an RNA virus such as a retrovirus. Preferably, the retroviral vector is a derivative of a murine or avian retrovirus. Examples of retroviral vectors in which a single foreign gene can be inserted include, but are not limited to Moloney murine leukemia virus (MoMuLV), Harvey marine sarcoma virus (HaMuS-V), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated.

Since recombinant retroviruses are defective, they require assistance in order to produce infectious vector particles. This assistance can be provided, for example, by using helper cell lines that contain plasmids encoding all of the structural genes of the retrovirus under the control of regulatory sequences within the LTR. These plasmids are missing a nucleotide sequence that enables the packaging mechanism to recognize an RNA transcript for encapsidation. Helper cell lines that have deletions of the packaging signal include, but are not limited to, ÿ, PA317 and PA12, for example. These cell lines produce empty virions, since no genome is packaged. If a retroviral vector is introduced into such helper cells in which the packaging signal is intact, but the structural genes are replaced by other genes of interest, the vector can be packaged and vector virion can be produced.

Certain advantages can be obtained by administering a polynucleotide encoding a peptide of the invention as a vaccine in lieu of administering the peptide as a traditional vaccine, including, for example, that the risk of potential toxicity such as anaphylactic shock associated with a proteinaceous vaccine is substantially avoided. Where contacted with a cell or administered to a subject, the polynucleotide or recombinant nucleic acid molecule of the invention, or vector containing the polynucleotide, can be administered as a “naked” DNA, or can be formulated into a delivery vehicle such as a liposome or colloidal particles, which can facilitate uptake of the polynucleotide and can reduce the likelihood of degradation of the polynucleotide prior to uptake by a cell.

A transformed cell or host cell generally refers to a cell (e.g., prokaryotic or eukaryotic) into which (or into an ancestor of which) has been introduced, by means of recombinant DNA techniques, a DNA molecule encoding a peptide of the invention, or analog thereof.

The present invention also provides a composition, which contains at least one peptide of the invention and can provide a plurality of different peptides of the invention, for example, a composition containing any of the peptides set forth as SEQ ID NOS: 1-16, or a composition containing any combination of such peptides. A composition of the invention generally is formulated in a physiologically acceptable solution and, if desired, can further contain one or more immunoadjuvants, for example, one or more cytokines, Freund's complete adjuvant, Freund's incomplete adjuvant, alum, or the like. Generally, where the composition contains one or more cytokines, the cytokines have an activity that is the same as or complements the inflammatory activity of the peptide of the invention. The composition also can contain any immunoadjuvant, including an immunostimulant or, if desired, an immunosuppressant, which can modulate the systemic immune response of an individual. Suitable substances having this activity are well known in the art and include IL-6, which can stimulate suppressor or cytotoxic T cells, and cyclosporin A and anti-CD4 antibodies, which can suppress the immune response. Such compounds can be administered separately or as a mixture with a vaccine of the invention.

A composition of the invention can be prepared for administration to a subject by mixing the immunogenic peptide or peptides with physiologically acceptable carriers. Such carriers will be nontoxic to recipients at the dosages and concentrations employed. Ordinarily, the preparation of such compositions entails combining the particular vaccine antigen with saline, buffers, antioxidants such as ascorbic acid, low molecular weight (less than about 10 residues) polypeptides, proteins, amino acids, carbohydrates including glucose or dextrans, or chelating agents such as EDTA, glutathione and other stabilizers and excipients. Such compositions can be in suspension, emulsion or lyophilized form and are formulated under conditions such that they are suitably prepared and approved for use in the desired application.

A physiologically acceptable carrier can be any material that, when combined with an immunogenic peptide or a polynucleotide of the invention, allows the ingredient to retain biological activity and does not undesirably disrupt a reaction with the subject's immune system. Examples include, but are not limited to, any of the standard physiologically acceptable carriers such as a phosphate buffered saline solution, water, emulsions such as oil/water emulsion, and various types of wetting agents. Preferred diluents for aerosol or parenteral administration are phosphate buffered saline or normal (0.9%) saline. Compositions comprising such carriers are formulated by well known conventional methods (see, for example, Remington's Pharmaceutical Sciences, Chapter 43, 14th Ed., Mack Publishing Co., Easton Pa. 18042, USA).

For administration to a subject, a peptide, or an encoding polynucleotide, generally is formulated as a composition. Accordingly, the present invention provides a composition, which generally contains, in addition to the peptide or polynucleotide of the invention, a carrier into which the peptide or polynucleotide can be conveniently formulated for administration. For example, the carrier can be an aqueous solution such as physiologically buffered saline or other solvent or vehicle such as a glycol, glycerol, an oil such as olive oil or an injectable organic esters. A carrier also can include a physiologically acceptable compound that acts, for example, to stabilize the peptide or encoding polynucleotide or to increase its absorption. Physiologically acceptable compounds include, for example, carbohydrates, such as glucose, sucrose or dextrans, antioxidants, such as ascorbic acid or glutathione, chelating agents, low molecular weight proteins or other stabilizers or excipients. Similarly, a cell that has been treated in culture for purposes of the practicing the methods of the invention, for example, synovial fluid mononuclear cells, dendritic cells, or the like, also can be formulated in a composition when the cells are to be administered to a subject.

It will be recognized to the skilled clinician, choice of a carrier, including a physiologically acceptable compound, depends, for example, on the manner in which the peptide or encoding polynucleotide is to be administered, as well as on the route of administration of the composition. Where the composition is administered under immunizing conditions, i.e., as a vaccine, it generally is administered intramuscularly, intradermally, or subcutaneously, but also can be administered parenterally such as intravenously, and can be administered by injection, intubation, or other such method known in the art. Where the desired modulation of the immune system is tolerization, the composition preferably is administered orally, or can be administered as above.

A composition of the invention also can contain a second reagent such as a diagnostic reagent, nutritional substance, toxin, or therapeutic agent, for example, a cancer chemotherapeutic agent. Preferably, the second reagent is an immunomodulatory agent, for example, an immunostimulatory agent such as a cytokine or a B7 molecule. In addition, where it is desired to stimulate an immune response, the composition can contain an adjuvant, for example, alum, DETOX adjuvant (Ribi Immunochem Research, Inc.; Hamilton Mont.), or Freund's complete or incomplete adjuvant. The addition of an adjuvant can enhance the immunogenicity of a peptide of the invention, thus decreasing the amount of antigen required to stimulate an immune response. Adjuvants can augment the immune response by prolonging antigen persistence, enhancing co-stimulatory signals, inducing granuloma formation, stimulating lymphocyte proliferation nonspecifically, or improving apposition of a T cell and an APC.

A composition comprising a peptide or polynucleotide of the invention also can be incorporated within an encapsulating material such as into an oil-in-water emulsion, a microemulsion, micelle, mixed micelle, liposome, microsphere or other polymer matrix (see, for example, Gregoriadis, Liposome Technology, Vol. 1 (CRC Press, Boca Raton, Fla. 1984); Fraley, et al., Trends Biochem. Sci., 6:77, 1981, each of which is incorporated herein by reference). Liposomes, for example, which consist of phospholipids or other lipids, are nontoxic, physiologically acceptable and metabolizable carriers that are relatively simple to make and administer. “Stealth” liposomes (see, for example, U.S. Pat. Nos. 5,882,679; 5,395,619; and 5,225,212, each of which is incorporated herein by reference) are an example of such encapsulating material. Cationic liposomes, for example, also can be modified with specific receptors or ligands (Morishita et al., J. Clin. Invest., 91:2580-2585, 1993, which is incorporated herein by reference). In addition, a polynucleotide agent can be introduced into a cell using, for example, adenovirus-polylysine DNA complexes (see, for example, Michael et al., J. Biol. Chem. 268:6866-6869, 1993, which is incorporated herein by reference).

The term “therapeutically effective amount” or “effective amount” means the amount of a compound or pharmaceutical composition that will elicit the biological or medical response of a tissue, system, animal or human that is being sought by the researcher, veterinarian, medical doctor or other clinician. Thus, the total amount of a composition to be administered in practicing a method of the invention can be administered to a subject as a single dose, either as a bolus or by infusion over a relatively short period of time, and can be followed up with one or more booster doses over a period of time. The amount of the composition to stimulate an immune response in a subject depends on various factors including the age and general health of the subject, as well as the route of administration and the number of treatments to be administered. In view of these factors, the skilled clinician will know to adjust the particular dosage as necessary. In general, the formulation of the composition and the routes and frequency of administration are determined, initially, using Phase I and Phase H clinical trials.

The terms “administration” or “administering” is defined to include an act of providing a compound or pharmaceutical composition of the invention to a subject in need of treatment. The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion. The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

The efficacy of a therapeutic method of the invention over time can be identified by an absence of symptoms or clinical signs of an immunological disorder in a subject predisposed to the disorder, but not yet exhibiting the signs or symptoms of the disorder at the time of onset of therapy. In subjects diagnosed as having the immunological disorder, or other condition in which it is desirable to modulate the immune response, the efficacy of a method of the invention can be evaluated by measuring a lessening in the severity of the signs or symptoms in the subject or by the occurrence of a surrogate end-point for the disorder. One skilled in the art will be able to recognize and adjust the therapeutic approach as needed. Accordingly, the invention is also directed to methods for monitoring a therapeutic regimen for treating a subject having CMV infection.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE 1

### Epitopic Nature of Cytomegalovirus Peptides

This example demonstrates the ability to identify pan-major histocompatibility complex (MHC) binder dominant CMV epitopes that induce proinflammatory immune responses in human T effectors ex vivo.

These experiments focus on confirming the in vitro recognition of the novel class I and class II CMV epitopes predicted to be strong MHC binders by validated computer algorithms using T cells from CMV-positive healthy donors. Phenotypical analysis of CD4+ T cell effectors included a marker of activation (CD69) as well as T cell inflammatory cytokines (TNFα, IFNγ, and IL-2). Functional analysis of CD4+ and CD8+ T cell effectors was measured by proliferative epitope-specific responses via ³H incorporation and IFNγ expression measured by ELISA, respectively.

Of the class I peptides, CMV65₁₈₂₋₁₉₀ (YTSAFVFPT) (SEQ ID NO: 9) was the most immunogenic, producing a mean of 1999 pg/ml of IFNγ measured by ELISA.

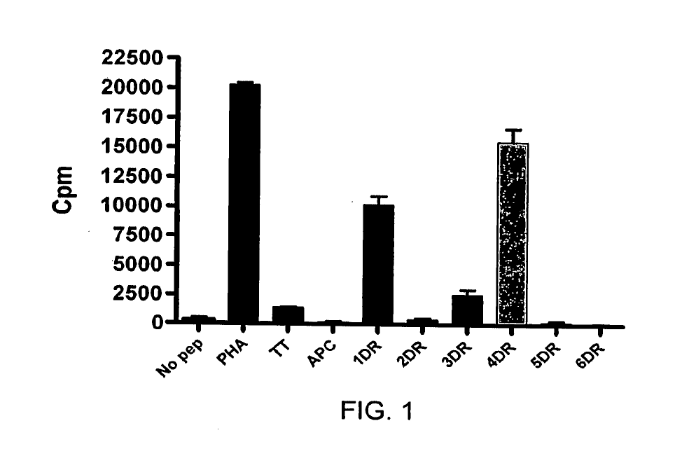

For the Class II peptides, CMV65₁₀₉₋₁₂₃ (1DR—MSIYVYALPLKMLNI) (SEQ ID NO: 11) and CMV65₁₇₉₋₁₉₃ (4DR—DVYYTSAFVFPTKDV) (SEQ ID NO: 14) were demonstrated the ability to be epitopic, producing 10,201 and 15,572 counts per minute (cpm), respectively. FIG. 1 shows data from a proliferation assay of CD4+ induced cells in which antigen-stimulated T effector cells are incubated with autologous, irradiated total PBMCs for 96 hours. Wells are then pulsed with 1 μCi of ³H for the last 18 hours. Proliferation is then measured by calculating the absorption of ³H on a beta counter. PHA is a positive control while APCs alone are the negative control. This translates into stimulation indices of 30 and 45.8 in antigen-specific proliferation assays as compared to cells without peptide.

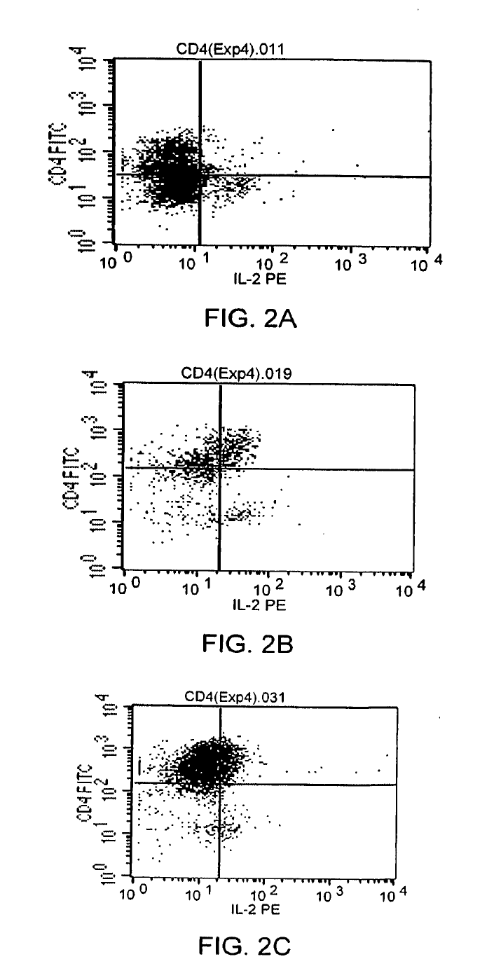

The immunogenicity of these Class II peptides was further corroborated by increased expression of IL-2 in CD4+ cells as compared to cells incubated without peptides, with 39.58% for CMV65₁₀₉₋₁₂₃ (1DR—MSIYVYALPLKMLNI) (SEQ ID NO: 11), 15.13% for CMV65₁₇₉₋₁₉₃ (4DR—DVYYTSAFVFPTKDV) (SEQ ID NO: 14), and 3.18% for cells without peptide (FIG. 2).

FIG. 2 shows data that peptides 1DR and 4DR stimulate expression of IL-2 as compared to no peptide. Enriched CD4+ cells are incubated with peptide-pulsed irradiated autologous PBMCs, restimulated with peptide on day 7, stimulated with low-dose IL-2 (20 U/ml) on day 9, and then evaluated for intracellular cytokine production on day 17. FIG. 2A illustrates no peptide, while FIGS. 2B and 2C show stimulation by peptides 1DR and 4DR, respectively.

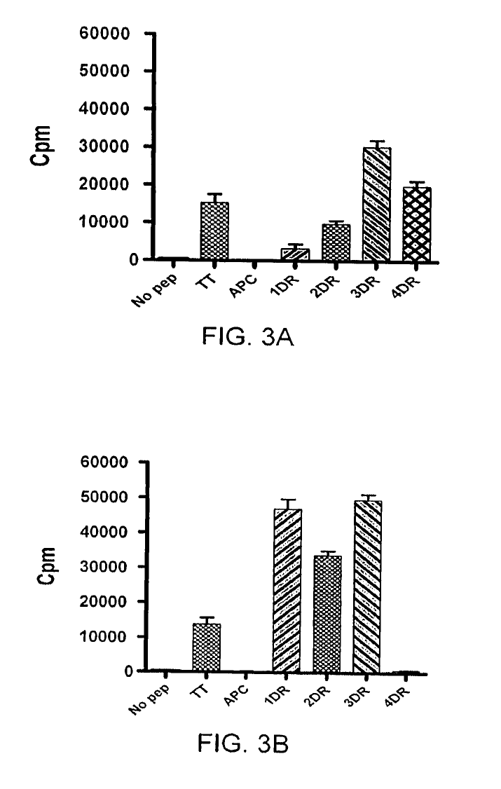

For the class II peptides, CMV65₄₈₋₆₂ (3DR—HVRVSQPSLILVSQY) (SEQ ID NO: 13) was also epitopic, producing 30,231.67 and 49,648.33 counts per minute (cpm) in the two CMV+ healthy donors shown below (FIG. 3). This translates into stimulation indices of 83.05 and 147.03 in antigen-specific proliferation assays as compared to cells without peptide.

FIG. 3 shows data from a proliferation assay of CD4 Induced Cells in Donors of Different HLA Alleles. Antigen-stimulated T effector cells are incubated with autologous, irradiated total peripheral blood mononuclear cells for 96 hours. Wells are pulsed with 1 μCi of ³H for the last 18 hours. Proliferation is then measured by calculating the absorption of ³H on a beta counter. APCs alone are the negative control. FIG. 3A shows Donor SA005 HLA DRB1 1/1, while FIG. 3B shows Donor SA006 HLA DRB1 1/11.

Of note, donor SA005 is HLA DRB1 1/13 while donor SA006 is HLA DRB1 1/11. This demonstrates the concept in these two donors that a peptide predicted by the matrix-binding algorithms can be epitopic in multiple HLA alleles. In addition, these experiments also demonstrate that the other peptides that have been chosen are also epitopic in these donors.

The immunogenicity of these class II peptides was further corroborated by increased expression of TNFα in CD4 cells as compared to cells incubated without peptides, with 15.9% for CMV65₁₀₉₋₁₂₃ (1DR—MSIYVYALPLKMLNI) (SEQ ID NO: 11) in donor SA005 and 34.58% in CMV65₄₈₋₆₂ (3DR—HVRVSQPSLILVSQY) (SEQ ID NO: 13) (FIG. 3A). In addition, these cells are all chemokine receptor CCR7 negative, reflecting their T effector status.

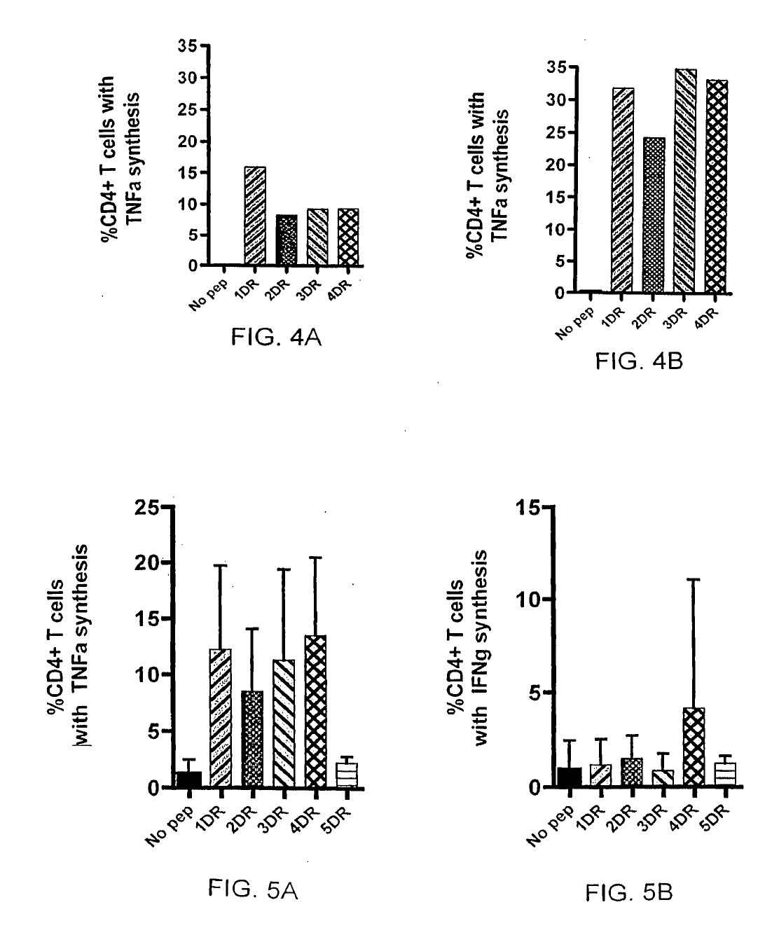

FIG. 4 shows data demonstrating that peptides 1DR and 4DR stimulate TNFα synthesis as compared to control. Enriched CD4 cells are incubated with peptide-pulsed irradiated autologous peripheral blood mononuclear cells, restimulated with peptide on day 7, stimulated with low-dose IL-2 (20 U/ml) on day 10 and 14, and then evaluated for intracellular cytokine production on day 17. FIG. 4A shows Donor SA005 HLA DRB1 1/1, while FIG. 4B shows Donor SA006 HLA DRB1 1/11.

FIGS. 5A and 5B are graphical diagrams showing the immunogenicity of peptides 1DR to 4DR in two healthy CMV+ donors. FIG. 5A shows Donor SA005 HLA DRB1 1/1, while FIG. 5B shows Donor SA006 HLA DRB1 1/11.

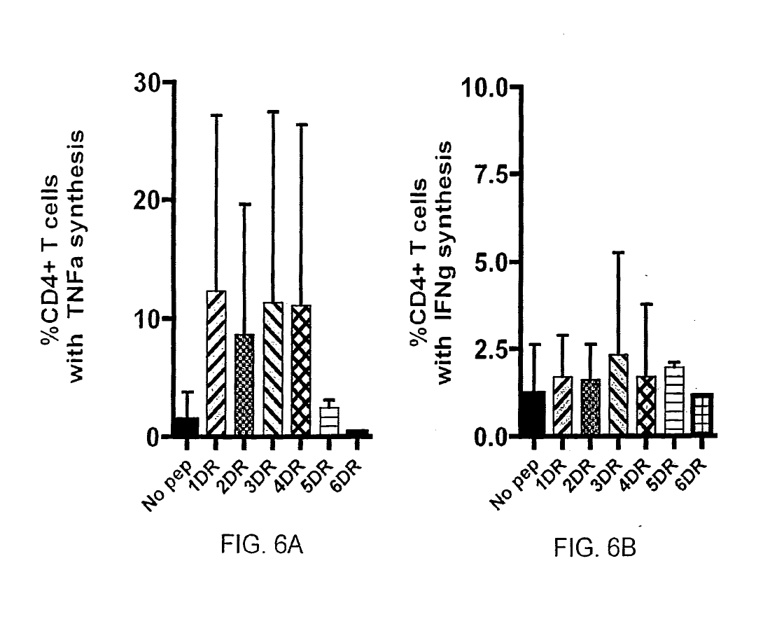

FIGS. 6A and 6B are graphical diagrams showing the immunogenicity of peptides 1DR to 4DR in four healthy CMV+ donors.

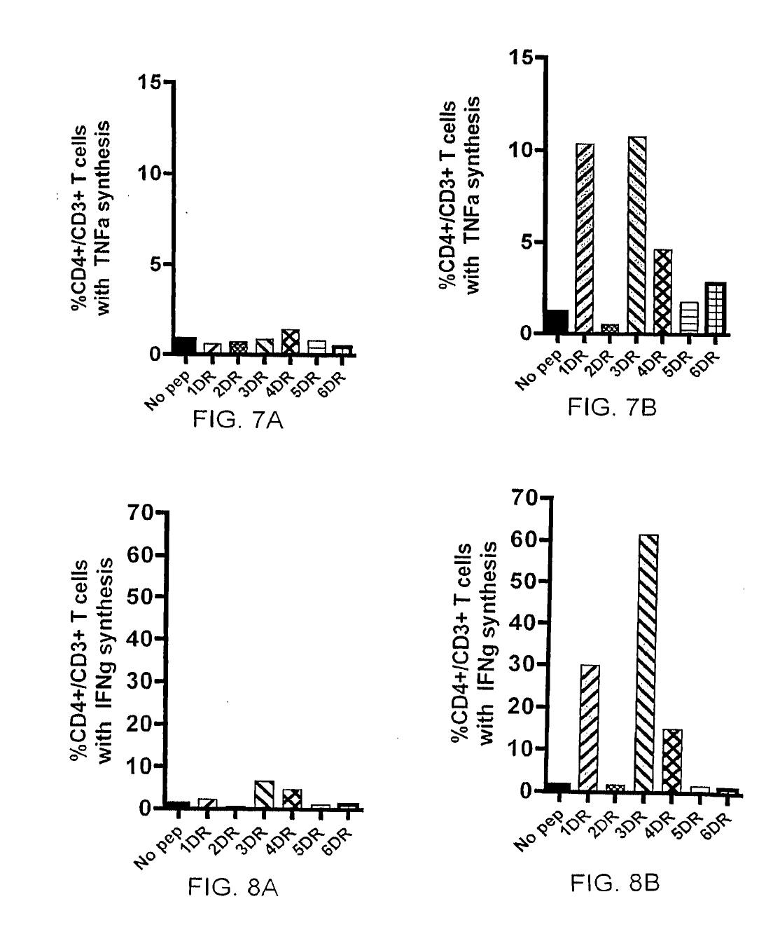

FIGS. 7A and 7B are graphical diagrams showing that the immunogenicity (reflected by increased TNFα) of peptides 1DR, 3DR and 4DR in a CMV-infected subject (CMV001) is greater than in a healthy CMV+ subject (BB008). FIG. 7A shows Donor BB008, while FIG. 7B shows Donor CMV001.

FIGS. 8A and 8B are graphical diagrams showing that the immunogenicity (reflected by increased IFNg) of peptides 1DR, 3DR and 4DR in a CMV-infected subject (CMV001) is greater than in a healthy CMV+ subject (BB008). FIG. 8A shows Donor BB008, while FIG. 8B shows Donor CMV001.

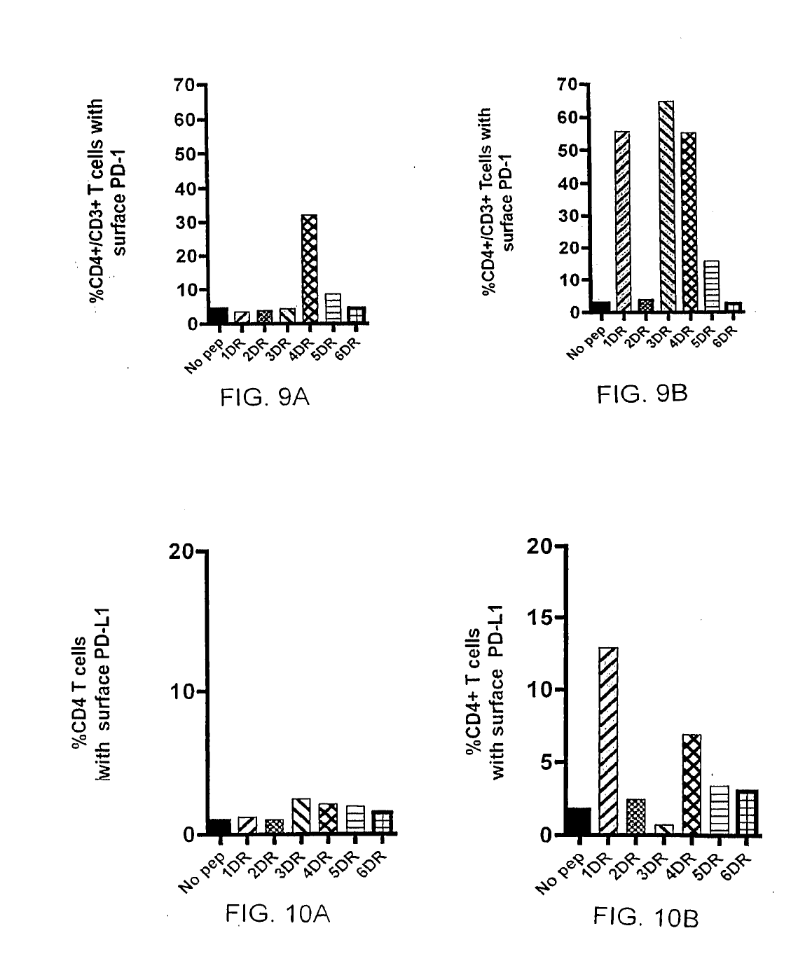

FIGS. 9A and 9B are graphical diagrams showing that there is more surface expression of PD-1 (programmed cell death-1) with peptides 1DR, 3DR and 4DR in a CMV-infected subject (CMV001) is greater than in a healthy CMV+ subject (BB008). This correlates with latency versus infection in the two subjects. FIG. 9A shows Donor BB008, while FIG. 9B shows Donor CMV001.

FIGS. 10A and 10B are graphical diagrams showing that there is more surface expression of PD-L1 (a PD-1 ligand) with peptides 1DR, 4DR, 5DR and 6DR in a CMV-infected subject (CMV001) is greater than in a healthy CMV+ subject (BB008). FIG. 10A shows Donor BB008, while FIG. 10B shows Donor CMV001.

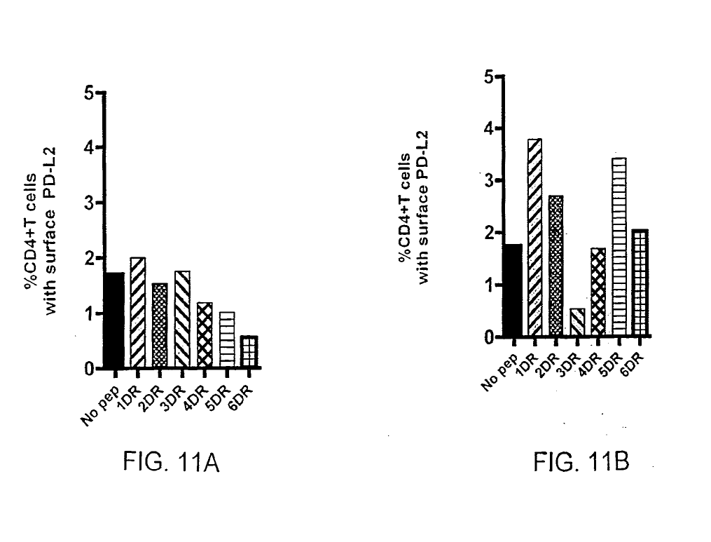

FIGS. 11A and 11B are graphical diagrams showing that there is more surface expression of PD-L2 (a PD-1 ligand) with peptides 1DR, 4DR, 5DR and 6DR in a CMV-infected subject (CMV001) is greater than in a healthy CMV+ subject (BB008). FIG. 11A shows Donor BB008, while FIG. 11B shows Donor CMV001.

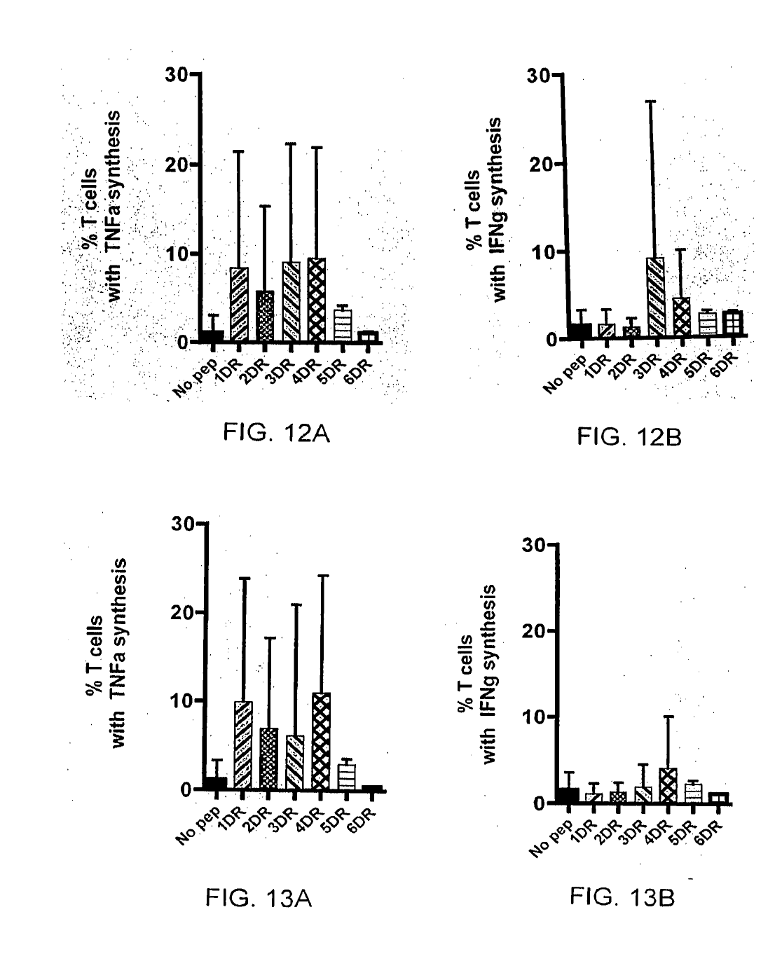

FIGS. 12A and 12B are graphical diagrams showing that the immunogenicity of peptides 1 DR, 3DR, and 4DR (reflected by increased TNFα and IFNg) in an experiment with six donors, excluding CMV-infected subject (CMV001).

FIGS. 13A and 13B are graphical diagrams showing that the immunogenicity of peptides 1DR and 4DR (reflected by increased TNFα and IFNg) in an experiment with six donors, including CMV-infected subject (CMV001). Removal of the CMV-infected donor appeared to affect 3DR IFNg but did not significantly impact statistical differences.

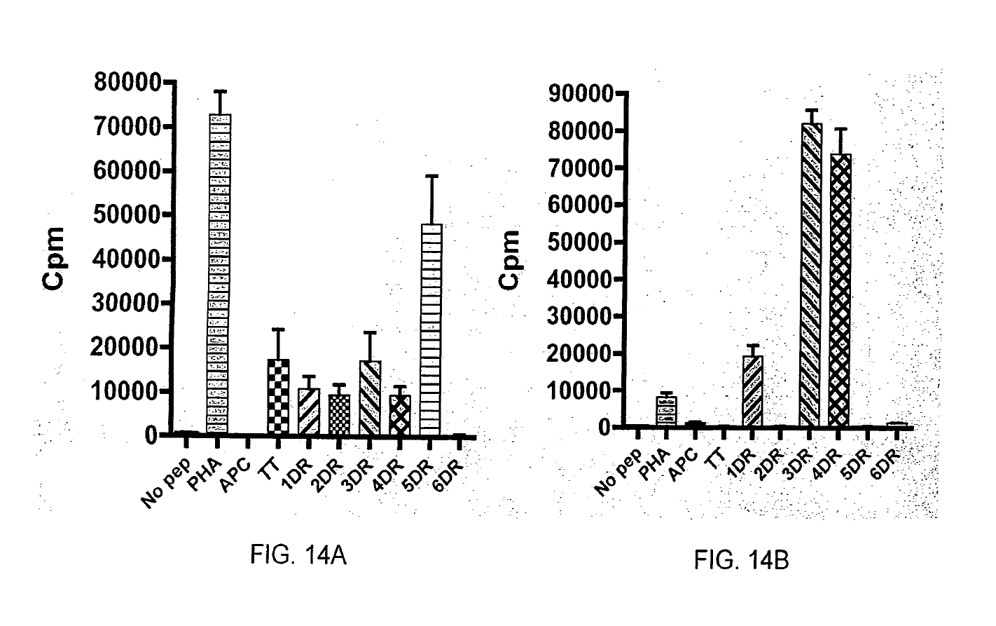

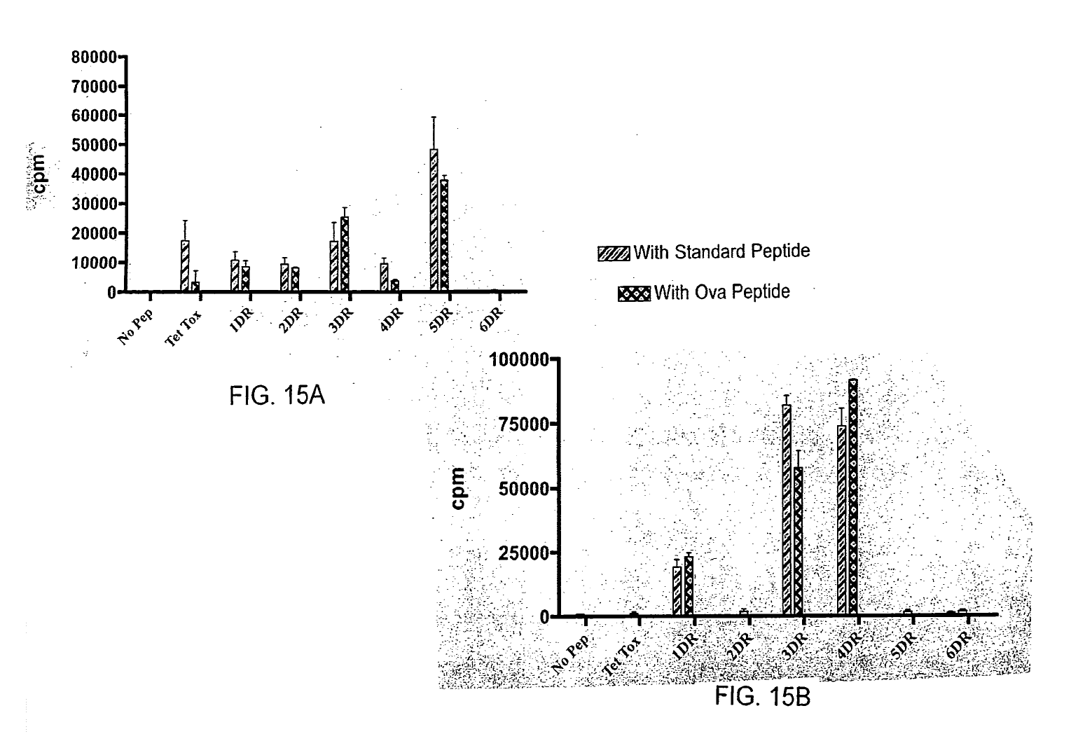

FIGS. 14A and 14B show data from a proliferation assay of cells from various donors using methods known in the art. FIG. 14A shows cells from donor BB008, while FIG. 14B shows cells from donor CMV001.

FIGS. 15A and 15B show data from a proliferation assay of cells from various donors using methods known in the art. FIG. 15A shows cells from donor BB008 with standard peptides 1DR-6DR and with Ova peptide, while FIG. 15B shows cells from donor CMV001 with standard peptides 1DR-6DR and with Ova peptide.

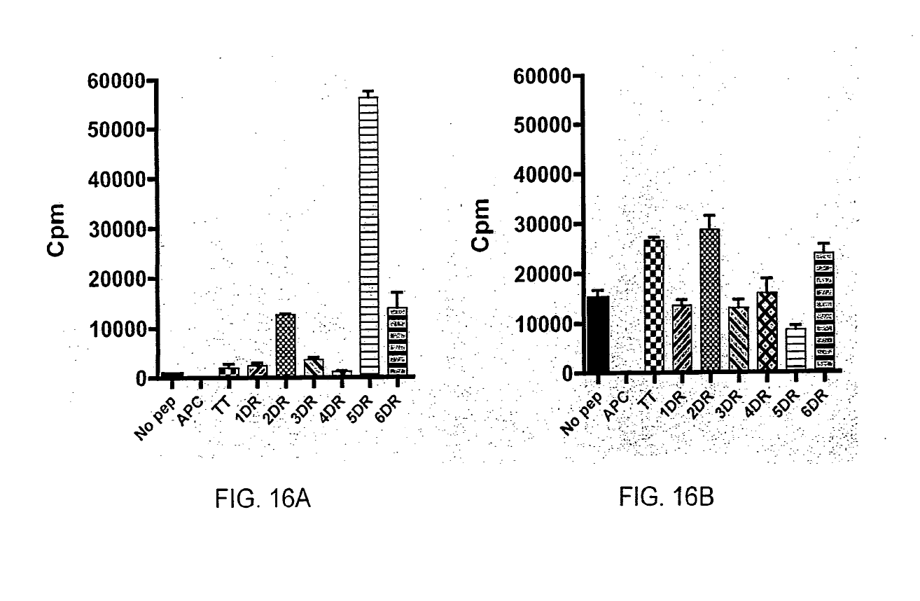

FIGS. 16A and 16B show data from a proliferation assay of cells from various donors using methods known in the art. FIG. 16A shows cells from donor BB011 with peptides 1DR-6DR, while FIG. 16B shows cells from donor BB013 with peptides 1DR-6DR.

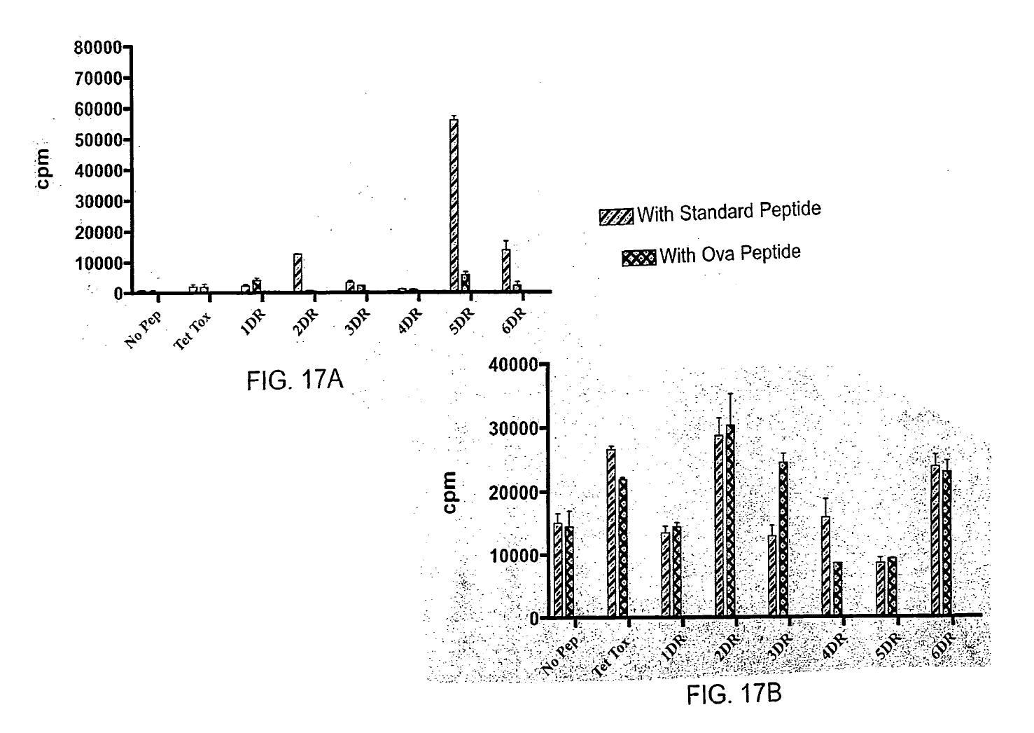

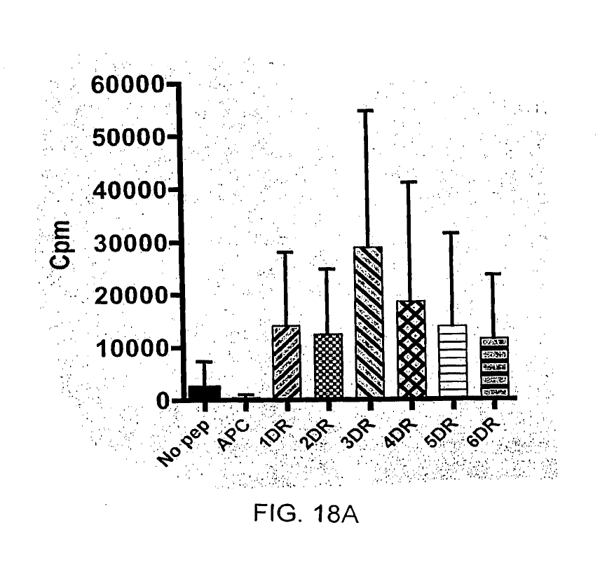

FIGS. 17A and 17B show data from a proliferation assay of cells from various donors using methods known in the art. FIG. 17A shows cells from donor BB011 with standard peptides 1 DR-6DR and with Ova peptide, while FIG. 17B shows cells from donor BB013 with standard peptides 1DR-6DR and with Ova peptide.

FIG. 18 shows data from a proliferation assay of cells from various donors using methods known in the art. Data from the six donors SA001, SA005, SA006, BB006, BB008, CMV001, BB011, and BB013 are shown. No peptide versus 3DR (P<0.001); no peptide versus 4DR (P<0.05); 1DR versus 3DR (P<0.05); 2DR versus 3DR (P<0.01); and 3DR versus 6DR (P<0.05).

TABLE 2
PD-1 Taqman (Donors BB008 & CMV001)

| | ΔCT | | | |
|---|---|---|---|---|
| | (BB008) | ΔΔCT(BB008) | ΔCT(CMV001) | ΔΔCT(CMV001) |
| No Pep | 6.4 | 0 | 5.08 | 0 |
| 1DR | 5.385 | −1.015 | 3.635 | −1.445 |
| 2DR | 5.525 | −0.875 | 5.005 | −0.075 |
| 3DR | 5.635 | −0.765 | 2.545 | −2.535 |
| 4DR | 1.185 | −5.215 | 1.165 | −3.915 |
| 5DR | 6.26 | −0.14 | 3.53 | −1.55 |
| 6DR | — | — | — | — |

TABLE 3
PD-1 Taqman (Donors BB011 & BB013)

| | ΔCT | | | |
|---|---|---|---|---|
| | (BB011) | ΔΔCT(BB011) | ΔCT(BB013) | ΔΔCT(BB013) |
| No Pep | 3.57 | 0 | 4.745 | 0 |
| 1DR | 3.67 | 0.1 | 5.74 | 0.995 |
| 2DR | 2.94 | −0.63 | 5.26 | 0.515 |
| 3DR | 4.08 | 0.51 | 5.995 | 1.25 |
| 4DR | 3.345 | −0.225 | 4.57 | −0.175 |
| 5DR | 3.97 | 0.4 | 6.73 | 1.985 |
| 6DR | 3.845 | 0.275 | 5.05 | 0.305 |

These preliminary results demonstrate the epitopic nature of these novel class I and II peptides, supporting the immunogenicity of the proposed epitopes. Furthermore, these studies have given us the opportunity to optimize these in vitro culture conditions which will be instrumental in the completion of this work.

FACS data suggest a peptide-specific TNFα and IFNg stimulation by peptides 1DR, 3DR and 4DR over multiple HLA-type donors. Proliferation data suggests that peptide 3DR leads to a functional response. Taqman data suggests PD-1 presence with the peptides.

Although the invention has been described with reference to the above example, it will be understood that modifications and variations are encompassed within the spirit and scope of the invention. Accordingly, the invention is limited only by the following claims.

What is claimed is:

1. An isolated peptide selected from any one of SEQ ID NOS: 1-16.

2. A chimeric polypeptide, comprising the peptide of claim 1 operatively linked to at least one heterologous polypeptide.

3. A pharmaceutical composition, comprising at least one peptide of claim 1.

4. The pharmaceutical composition of claim 3, comprising a plurality of peptides.

5. The pharmaceutical composition of claim 4, which further comprises a pharmaceutically acceptable solution.

6. The pharmaceutical composition of claim 3, which further comprises an immunoadjuvant.

7. The pharmaceutical composition of claim 6, wherein the immunoadjuvant comprises Freund's complete adjuvant, Freund's incomplete adjuvant, or alum.

8. An isolated polynucleotide encoding a peptide of claim 1.

9. An isolated nucleic acid molecule, comprising the polynucleotide of claim 8 operatively linked to at least one heterologous nucleotide sequence.

10. The nucleic acid molecule of claim 9, wherein the heterologous nucleotide sequence comprises a transcription regulatory element, a translation regulatory element, or a combination thereof.

11. The nucleic acid molecule of claim 9, wherein the heterologous nucleotide sequence encodes a polypeptide.

12. The nucleic acid molecule of claim 11, wherein the polypeptide is a cytokine.

13. The nucleic acid molecule of claim 11, wherein the polypeptide is selected from the group consisting of SEQ ID NOS: 1-16 and a combination thereof.

14. The nucleic acid molecule of claim 13, heterologous nucleotide sequence further encodes a protease recognition site between each of the encoded polypeptides.

15. A vector, which contains the polynucleotide of claim 8.

16. The vector of claim 15, wherein the vector is a plasmid vector.

17. The vector of claim 15, wherein the vector is a viral vector.

18. An isolated host cell stably transformed with the vector of claim 15.

19. A cell which contains the polynucleotide of claim 8.

20. A method of stimulating an immune response in a subject having or at risk of having cytomegalovirus infection, comprising administering to the subject an isolated peptide selected from any one of SEQ ID NOS: 1-16 and any combination thereof, thereby stimulating an immune response to the cytomegalovirus infection in the subject.

21. The method of claim 20, wherein the peptide is glycosylated.

22. A method of stimulating an immune response in a subject having or at risk of having cytomegalovirus infection, comprising contacting ex vivo a sample of cells from the subject with an isolated peptide selected from any one of SEQ ID NOS: 1-16 and any combination thereof, and subsequently administering the contacted cells to the subject, thereby stimulating an immune response to the cytomegalovirus infection in the subject.

23. An in vitro method for identifying an agent that enhances stimulation of an immune response in a subject having or at risk of having cytomegalovirus infection comprising contacting a sample comprising cells that express a detectable marker with a test agent and an isolated peptide selected from any one of SEQ ID NOS: 1-16, wherein an increase in the expression of the detectable marker in the presence of the agent as compared with expression of the detectable marker in the absence of the agent is indicative of an agent that enhances stimulation of an immune response in a subject having or at risk of having cytomegalovirus infection.

24. The method of claim 23, wherein the marker is CD69.

25. The method of claim 23, wherein the marker is a cytokine selected from the group consisting of TNFα, IFNγ, and IL-2.
